(19) United States
(12) Patent Application Publication
(10) Pub. No.: US 2025/0057801 A1
(43) Pub. Date: Feb. 20, 2025

(54) Methods for Treating Traumatic Brain Injury

(71) Applicant: The Government of the United States, as Represented by the Secretary of the Army, Fort Detrick, MD (US)

(72) Inventors: Jignesh D. Pandya, Germantown, MD (US); Deborah Shear, Columbia, MD (US); Hiren Modi, Germantown, MD (US); Sudeep Musyaju, Olney, MD (US); Anke H. Scultetus, Chester, MD (US)

(21) Appl. No.: 18/794,153

(22) Filed: Aug. 5, 2024

Related U.S. Application Data
(60) Provisional application No. 63/532,457, filed on Aug. 14, 2023.

(51) Int. Cl. A61K 31/222; A61K 31/22; A61K 31/221; A61K 31/66; A61P 25/28; A61P 39/06

(52) U.S. Cl. CPC A61K 31/222; A61K 31/22; A61K 31/221; A61K 31/66; A61P 25/28; A61P 39/06

(57) ABSTRACT

A method for treating traumatic brain injury include administering to a patient having traumatic brain injury and in need of treatment an effective amount of a composition comprising resveratrol triacetate or esterified resveratrol. The traumatic brain injury may be a penetrating brain injury.

This application claims priority to U.S. Ser. No. 63/532,457 filed in the US Patent and Trademark Office on 14 Aug. 2023, the entirety of which is incorporated herein by reference.

### FIELD OF INVENTION

The present invention is directed to methods for treating traumatic brain injury with a composition comprising resveratrol triacetate (triacetyl resveratrol or RES). In a specific embodiment, the traumatic brain injury may be a penetrating traumatic brain injury.

### BACKGROUND OF INVENTION

Resveratrol is a natural compound present in grapes, mulberries, peanuts, and in several other plants. This compound has low water solubility and short half-life (in 8-14 minutes). Therefore, many strategies have been employed to increase its water solubility and extend half-life, so that the drug remains longer in circulation to achieve higher therapeutic efficacy.

Triacetyl resveratrol is formulated by addition of acetyl moieties to resveratrol with the result that it displays greater bioavailability (absorption), solubility and potential to extend half-life compared to the parent compound. Thus, triacetyl resveratrol (3′,5′,4′-tri-o-acetyl resveratrol) is a cell-permeable and stable resveratrol prodrug. It has been hypothesized that the three acetate moieties may be able to provide energy to an injured site; whereas, resveratrol may be able to provide other cytoprotective effects (e.g., antioxidant, anti-inflammatory, and antiapoptotic) after brain trauma. Triacetyl resveratrol can be easily digested to the parent compound resveratrol by esterase and distributes rapidly to tissues within the body.

Studies have shown that resveratrol has several beneficial effects that include decreasing inflammation, reducing cell death activity, anti-cancer activity, and activity as a powerful antioxidant to mitigate the effects of oxidative stress. Oxidative stress is an imbalance of free radicals and antioxidants in the body, more specifically in the mitochondria (intracellular organelle), which can further lead to cell and tissue damage.

Among the factors leading to traumatic brain injury (TBI) outcome are biochemical cascades which occur in response to primary and secondary injury. These mechanisms elevate oxidative stress, and decrease endogenous antioxidant level, that can result in neural dysfunction and death.

Military service members and veterans are at risk of traumatic brain injury from explosions experienced during combat or training exercises. TBI is one of the signature injuries during military operations that lack approved treatment. Depending on the severity of the brain injury, a person with TBI may experience a change in consciousness that can range from being dazed and confused to losing consciousness. Secondary injury is progressive following direct insult. Mitochondrial dysfunction contributes to TBI pathogenesis. Currently, neuroprotective therapeutic intervention for acute TBI is an unmet need.

Resveratrol has been the subject of a study; however, there has been no clinical study for resveratrol derivatives or triacetyl resveratrol for treating traumatic brain injury (TBI).

### SUMMARY OF INVENTION

The present invention provides in a first embodiment a method for treating traumatic brain injury comprising administering to a patient having traumatic brain injury and in need of treatment an effective amount of a composition comprising resveratrol triacetate (RES).

The invention provides in a second embodiment further to any of the previous embodiments a method for treating traumatic brain injury comprising administering to the patient about 50 mg/kg/day to about 150 mg/kg/day of resveratrol triacetate.

The invention provides in a third embodiment further to any of the previous embodiments a method for treating traumatic brain injury wherein the administering is by an oral supplement or additive. The administering may also be by a nasal spray or intramuscular injection.

The invention provides in a fourth embodiment further to any of the previous embodiments a method for treating traumatic brain injury wherein the administration initiated within about 15 minutes to about 60 minutes, and may continue to be given once daily for a sub-acute period (e.g., 0-14 days) after a traumatic brain injury event.

The invention provides in a fifth embodiment further to any of the previous embodiments a method for treating traumatic brain injury further comprising administering to the patient an effective amount of at least one of acetyl-L-carnitine, glyceryl triacetate, or mitoquinone.

The invention provides in a sixth embodiment further to any of the previous embodiments a method for treating traumatic brain injury wherein the patient has acute, sub-acute, and/or chronic post-traumatic brain injury.

The invention provides in a seventh embodiment further to any of the previous embodiments a method for treating traumatic brain injury wherein the administering preserves or increases mitochondrial antioxidant levels in the patient.

The invention provides in an eighth embodiment further to any of the previous embodiments a method for treating traumatic brain injury wherein the traumatic brain injury comprises a penetrating brain injury.

### BRIEF DESCRIPTION OF THE DRAWINGS

FIGS. 1A-1F show the therapeutic efficacy of resveratrol triacetate (RES) treatment at 24 hours post-penetrating brain injury (PTBI): (A) Effect of RES treatment on bioenergetics in injured core mitochondria compared to vehicle (VEH); (B) RES administration reversed PTBI-induced downregulation of total glutathione (GSH) content; (C) A western blot image showing the expression of antioxidant markers; (D-F) RES treatment maintains homeostasis of a first line of antioxidant defense compared to VEH.

FIGS. 2A-2C show the therapeutic efficacy of resveratrol triacetate (RES) treatment at 24 hours post-penetrating brain injury (PTBI): (A, C) Effect of RES treatment in membrane integrity and apoptosis markers following PTBI compared to VEH; (B) Such effects were accompanied by non-significant improvement in the lipid peroxidation marker, 4-hydroxynonenal (4-HNE) adduct formation, compared to VEH.

FIGS. 3A-3D show the therapeutic efficacy of RES treatment at 3 days post-PTBI: (A) Effect of RES on bioenergetics in injured core mitochondria compared to VEH; (B) A western blot image showing the expression of antioxidants; (C-D) RES treatment significantly maintained antioxidant defense expression compared to VEH.

FIGS. 4A-4D shows the effect of RES on membrane integrity and apoptosis markers at 3 days post-PTBI: (A) A western blot image showing the expression of various markers; (B-C) RES treatment preserved mitochondrial membrane proteins, cytochrome C and voltage-dependent anion channels compared to VEH; (D) RES treatment improved the expression of anti-apoptotic protein Bcl-2 compared to VEH.

### DETAILED DESCRIPTION OF THE INVENTION

The present invention is directed to methods for treating traumatic brain injury (TBI) with a composition comprising resveratrol triacetate or triacetyl resveratrol. In a specific embodiment, the traumatic brain injury may be a penetrating traumatic brain injury (PTBI). Resveratrol triacetate may prevent the detrimental acute and sub-acute mitochondrial responses following PTBI. Salient features of PTBI may include a permanent brain cavity caused, for example, by a bullet path, or a temporary cavity caused, for example, by inward air movement and/or shock. In another embodiment, a method for treating traumatic brain injury (TBI) may include administering an effective amount of a composition comprising an esterified form of resveratrol.

In this detailed description, references to “one embodiment”, “an embodiment”, or “in embodiments” mean that the feature being referred to is included in at least one embodiment of the invention. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

As used herein “substantially”, “generally”, “about”, and other words of degree are relative modifiers intended to indicate permissible variation from the characteristic so modified (e.g., +0.1%, +0.5%, +1.0%, +2%, +5%, +10%, +20%). It is not intended to be limited to the absolute value or characteristic which it modifies but rather possessing more of the physical or functional characteristic than its opposite, and preferably, approaching or approximating such a physical or functional characteristic.

According to the present invention, a method for treating traumatic brain injury includes administering to a patient having traumatic brain injury and in need of treatment an effective amount of a composition comprising resveratrol triacetate. Resveratrol triacetate. In embodiments, the amount of RES administered is about 50 mg/kg to about 150 mg/kg per day, for example, about 100 mg/kg per day. However, efficacy has been shown in a preclinical rodent model following PTBI. The actual efficacy may differ when treating human TBI or PTBI.

In specific embodiments, administration of the composition may be by any effective means, for example, by oral supplement, additive, or injection (e.g., intramuscular injection). In embodiments, the administration may be within about 15 minutes to about 60 minutes of a traumatic brain injury event. The administration may continue to be given once daily for a sub-acute period (e.g., 0-14 days) after a traumatic brain injury event.

In embodiments, the composition may comprise or may be administered with an additional compound including, but not limited to, an effective amount of at least one of acetyl-L-carnitine, glyceryl triacetate, or mitoquinone. These compounds have a beneficial effect in mitochondria following TBI.

According to the present invention, a patient may have acute, sub-acute, and/or chronic post-traumatic brain injury. In a specific embodiment, the traumatic brain injury comprises a penetrating brain injury or mild TBIs (e.g., concussive injury or any contact TBI injuries including sports TBI).

The inventors tested triacetyl resveratrol in an animal model of PTBI considering its bioavailability. The inventors measured the mitochondrial functions as well as antioxidant status for recovery following triacetyl resveratrol treatment. The mitochondrial bioenergetics showed a recovery trend with triacetyl resveratrol-treated group compared to a vehicle-treated group following PTBI. Post-PTBI, triacetyl resveratrol treatment significantly improved mitochondrial antioxidant levels. Additionally, observed cell death apoptotic markers were stabilized to normal level in the treatment group. This result provides evidence that triacetyl resveratrol has a protective role in TBI.

### Experimental Methods

Three experimental groups (N=4-8 male Sprague-Dawley rats/group) were studied. The first group was a sham group in which there was no penetrating brain injury and no treatment with resveratrol triacetate (RES). The second group was subject to a penetrating brain injury (via probe insertion and balloon inflation) and treated with dimethyl sulfoxide, DMSO (vehicle, VEH) and dosed at 15 minutes and 6 hours after injury and then once daily. The third group was subject to a penetrating brain injury and treated with RES and dosed at 15 minutes and six hours after injury and then once daily.

After 60 minutes of the last dosing at either 24 hours or 3 days post-injury, fresh viable mitochondria were isolated from the injury core of rats' brain. Homogenate fractions were collected for a glutathione assay.

Mitochondrial bioenergetics parameters were measured using a high-throughput procedure of the Seahorse XFe96 Flux Analyzer. The glutathione content was measured using a 5,5′-dithiobis-(2-nitrobenzoic acid) (DTNB) cycling method/assay. Antioxidant and oxidative stress were measured by a Western blot (WB) analysis.

Graphs and statistical analysis were performed using GraphPad Prism program. Data are presented as mean±SEM (standard error of means). ANOVA with Fisher post-hoc test was used to evaluate significant difference at * p<0.05 vs. Sham; $ p<0.05 vs. Vehicle (N=4-8 animals/group).

### Experimental Results

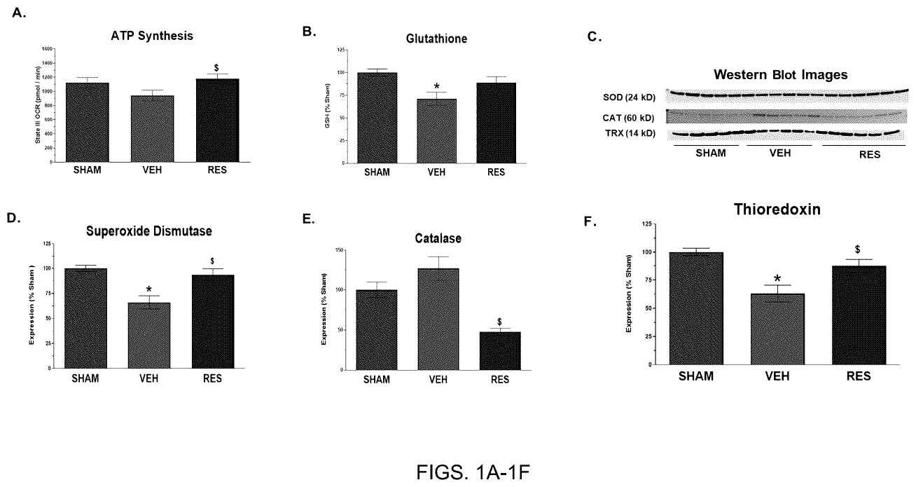

FIGS. 1A-1F show RES treatment rescued bioenergetics and antioxidant capacity at 24 hours following PTBI. FIG. 1A shows that RES treatment showed a trend of improved bioenergetics in injured core mitochondria, as measured by adenosine 5′-triphosphate (ATP) synthesis compared to vehicle (VEH) FIG. 1B shows that RES administration reversed PTBI-induced downregulation of total glutathione (GSH) content. GSH is an antioxidant produced throughout the body that plays many important biological roles, including neutralizing unstable oxygen molecules and provide protection against reactive oxygen and nitrogen species. FIG. 1C represents a western blot image showing the expression of antioxidant markers. FIGS. 1D-1F show that RES treatment maintained homeostatsis of a first line of antioxidant defense, superoxide dismutase (SOD +42%) and catalase (CAT −62%), along with thioredoxin (TRX +38%) following PTBI compared to the VEH. SOD, CAT and TRX are components of a natural antioxidant system that neutralize harmful effects of reactive oxygen species (ROS) and protect cellular homeostasis.

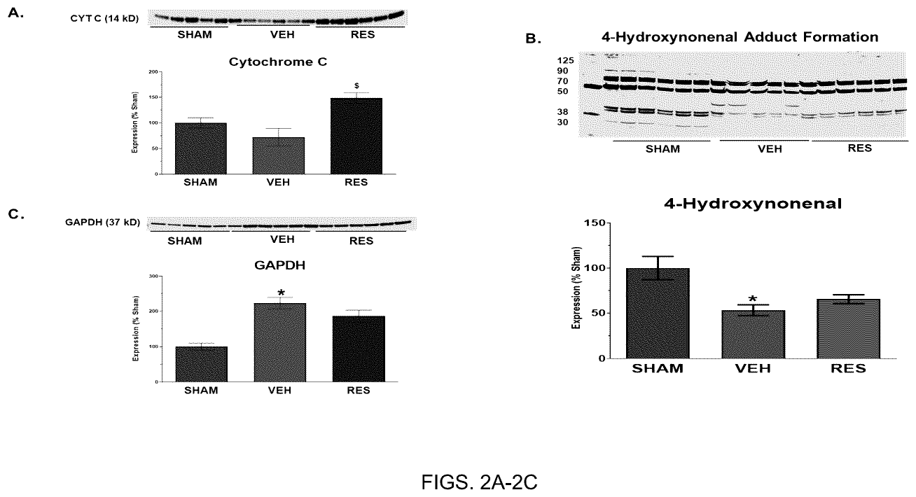

FIGS. 2A-2C show RES treatment showed improvement in the membrane integrity, apoptosis, and lipid peroxidation markers at 24 hours following PTBI. FIGS. 2A and 2C show that RES treatment preserved expressions of mitochondrial membrane protein Cytochrome C (CYT C, +105%) and apoptosis inducer glyceraldehyde 3-phosphate dehydrogenase (GAPDH, −16.5%) compared to the VEH. FIG. 2B shows that such effects were accompanied by non-significant improvement in the lipid peroxidation marker, 4-hydroxynonenal (4-HNE) adduct formation compared to the VEH. CYT C is an indicator of mitochondrial membrane integrity marker. GAPDH is considered as the apoptosis signaling marker. 4-HNE is a major end-product that is derived from the oxidation of n-6 poly unsaturated fatty acids (PUFAs).

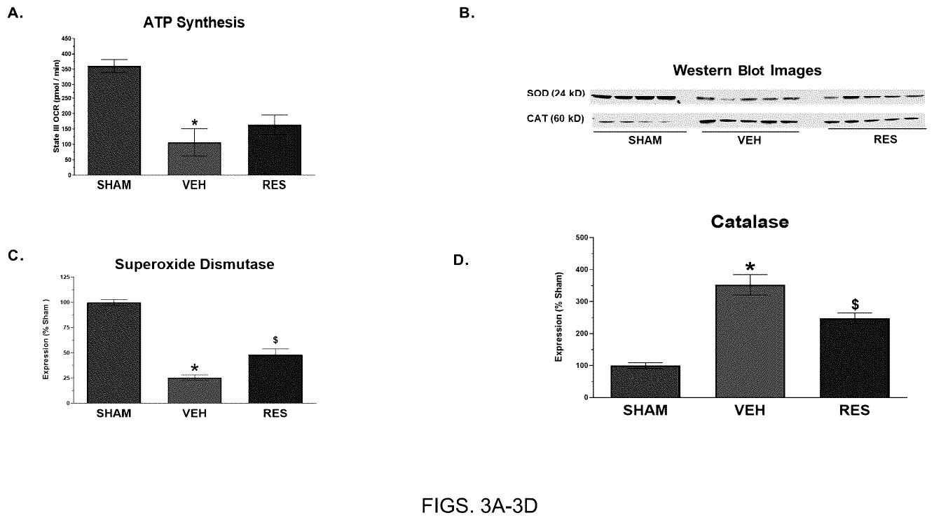

FIGS. 3A-3D show that RES treatment preserved bioenergetics and antioxidant capacity at 3 days following PTBI. FIG. 3A shows that RES treatment showed non-significant trend of bioenergetics improvement in injured core mitochondria compared to the VEH. FIG. 3B represents a western blot image showing the expression of antioxidants. FIGS. 3C-3D show that RES treatment significantly maintained expression of SOD (+90%) and CAT (−30%) following PTBI compared to the VEH.

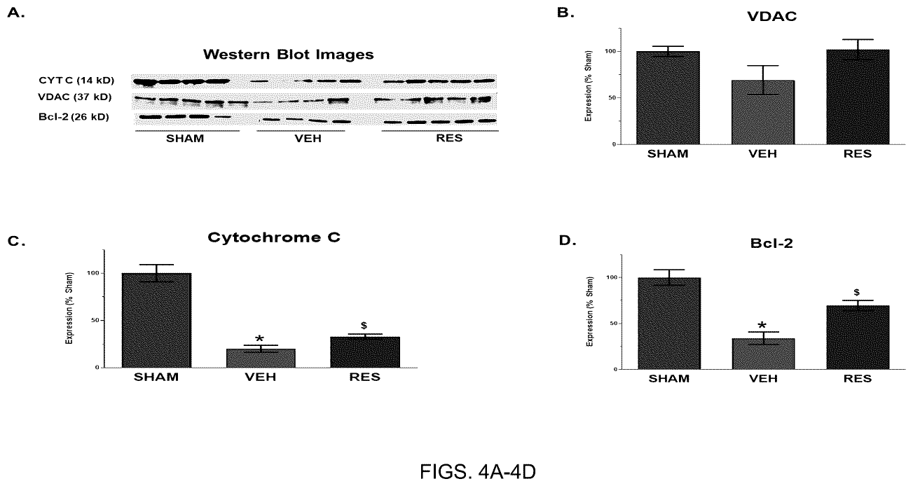

FIGS. 4A-4D show that RES treatment displayed improvement in membrane integrity and apoptosis markers at 3 days following PTBI. FIG. 4A represents a western blot image showing the expression of various markers. FIGS. 4B-4C show that RES treatment preserved mitochondrial membrane protein voltage-dependent anion channels (VDAC, +47%) and CYT C (+64%) compared to the VEH. FIG. 4D shows that RES treatment improved the expression of anti-apoptotic protein Bcl-2 (+104%) compared to the VEH. VDAC and CYT C are considered to be the mitochondrial membrane integrity markers which helps to maintain normal mitochondrial electron transport chain function and efficiency. Bcl-2 is an apoptosis marker, an indicator of cell death signaling during cellular stress conditions.

Although the present invention has been described in terms of particular exemplary and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

What is claimed is:

1. A method for treating traumatic brain injury, comprising:
administering to a patient having traumatic brain injury and in need of treatment an effective amount of a composition comprising resveratrol triacetate or esterified resveratrol.

2. The method of claim 1, comprising administering to the patient about 50 mg/kg to about 150 mg/kg per day of resveratrol triacetate.

3. The method of claim 1, wherein said administering is by an oral supplement or additive.

4. The method of claim 1, wherein said administering is by a nasal spray or intramuscular injection.

5. The method of claim 1, wherein said administering is within about 15 minutes to about 60 minutes after a traumatic brain injury event.

6. The method of claim 1, wherein said composition comprises resveratrol triacetate.

7. The method of claim 1, wherein said composition comprises esterified resveratrol.

8. The method of claim 1, further comprising administering to the patient an effective amount of at least one of acetyl-L-carnitine, glyceryl triacetate, or mitoquinone.

9. The method of claim 1, wherein the patient has acute, sub-acute, and/or chronic post-traumatic brain injury.

10. The method of claim 1, wherein said administering preserves or increases mitochondrial antioxidant levels in the patient.

11. The method of claim 1, wherein said traumatic brain injury comprises a penetrating brain injury.
